(19) United States
(12) Patent Application Publication
(10) Pub. No.: US 2011/0034983 A1
(43) Pub. Date: Feb. 10, 2011

(54) IMPLANTABLE MEDICAL DEVICE LEAD INCORPORATING A CONDUCTIVE SHEATH SURROUNDING INSULATED COILS TO REDUCE LEAD HEATING DURING MRI

(75) Inventors: Xiaoyi Min, Thousand Oaks, CA (US); Ingmar Viohl, Canyon Country, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/537,916

(22) Filed: Aug. 7, 2009

(51) Int. Cl. A61N 1/05

(52) U.S. Cl. 607/122; 607/116

(57) ABSTRACT

A conducting sheath is provided along at least a portion of an implantable medical device lead, and preferably along substantially its entire length, for mitigating heating problems arising during magnetic resonance imaging (MRI) procedures, particularly problems arising due to a problem described herein as the “coiling effect.” During device implant, the clinician may elect to wrap or coil excess proximal portions of leads around or under the medical device being implanted. Thereafter, during MRI procedures, shunt capacitance may develop between the housing of the implantable device and insulated coils within the proximal portions of the lead that are near the device, resulting in greater lead heating during the MRI. The conducting sheath helps suppress induced currents and also reduces or eliminates shunt capacitance. The conducting sheath may be, for example, formed using a metal mesh or a conducting polymer tube incorporating non-ferrous metal powders. The sheath may be formed in ¼ wavelength segments.

### CROSS-REFERENCE TO RELATED APPLICATIONS

This application is related to U.S. patent application Ser. No. ______, filed concurrently herewith, titled “Implantable Medical Device Lead Incorporating Insulated Coils Formed as Inductive Bandstop Filters to Reduce Lead Heating During MRI” (Attorney Docket A09P1042), which is incorporated by reference herein in its entirety.

### FIELD OF THE INVENTION

The invention generally relates to leads for use with implantable medical devices, such as pacemakers or implantable cardioverter-defibrillators (ICDs) and, in particular, to components for use within such leads to reduce heating during magnetic resonance imaging (MRI) procedures.

### BACKGROUND OF THE INVENTION

MRI is an effective, non-invasive magnetic imaging technique for generating sharp images of the internal anatomy of the human body, which provides an efficient means for diagnosing disorders such as neurological and cardiac abnormalities and for spotting tumors and the like. Briefly, the patient is placed within the center of a large superconducting magnetic that generates a powerful static magnetic field. The static magnetic field causes protons within tissues of the body to align with an axis of the static field. A pulsed RF magnetic field is then applied causing the protons to begin to precess around the axis of the static field. Pulsed gradient magnetic fields are then applied to cause the protons within selected locations of the body to emit RF signals, which are detected by sensors of the MRI system. Based on the RF signals emitted by the protons, the MRI system then generates a precise image of the selected locations of the body, typically image slices of organs of interest.

However, MRI procedures are problematic for patients with implantable medical devices such as pacemakers and ICDs. One of the significant problems or risks is that the strong RF fields of the MRI can induce currents through the lead system of the implantable device into the tissues, resulting in Joule heating in the cardiac tissues around the electrodes of leads and potentially damaging adjacent tissues. Indeed, the temperature at the tip or ring of an implanted lead has been found to increase as much as 60° degrees for tip or 20 degrees for ring Celsius (C) during an MRI tested in a gel phantom in a non-clinical configuration. Although such a dramatic increase is probably unlikely within a clinical system wherein leads are properly implanted, even a temperature increase of only about 8°-13° C. might cause myocardial tissue damage.

Furthermore, any significant heating of cardiac tissues near lead electrodes can affect the pacing and sensing parameters associated with the tissues near the electrode, thus potentially preventing pacing pulses from being properly captured within the heart of the patient and/or preventing intrinsic electrical events from being properly sensed by the device. The latter might result, depending upon the circumstances, in therapy being improperly delivered or improperly withheld. Another significant concern is that any currents induced in the lead system can potentially generate voltages within cardiac tissue comparable in amplitude and duration to stimulation pulses and hence might trigger unwanted contractions of heart tissue. The rate of such contractions can be extremely high, posing significant clinical risks to patients. Therefore, there is a need to reduce heating in the leads of implantable medical devices, especially pacemakers and ICDs, and to also reduce the risks of improper tissue stimulation during an MRI, which is referred to herein as MRI-induced pacing.

Various techniques have been developed to address these or other related concerns. See, for example, the following patents and patent applications: U.S. patent application Ser. No. 11/943,499, filed Nov. 20, 2007, of Zhao et al., entitled “RF Filter Packaging for Coaxial Implantable Medical Device Lead to Reduce Lead Heating during MRI”; U.S. patent application Ser. No. 12/117,069, filed May 8, 2008, of Vase, entitled “Shaft-mounted RF Filtering Elements for Implantable Medical Device Lead to Reduce Lead Heating During MRI”; U.S. patent application Ser. No. 11/860,342, filed Sep. 27, 2007, of Min et al., entitled “Systems and Methods for using Capacitive Elements to Reduce Heating within Implantable Medical Device Leads during an MRI”; U.S. patent application Ser. No. 12/042,605, filed Mar. 5, 2009, of Mouchawar et al., entitled “Systems and Methods for using Resistive Elements and Switching Systems to Reduce Heating within Implantable Medical Device Leads during an MRI”; and U.S. patent application Ser. No. 11/963,243, filed Dec. 21, 2007, of Vase et al., entitled “MEMS-based RF Filtering Devices for Implantable Medical Device Leads to Reduce Lead Heating during MRI.”

See, also, U.S. patent application Ser. No. 12/257,263, filed Oct. 23, 2008, of Min, entitled “Systems and Methods for Exploiting the Tip or Ring Conductor of an Implantable Medical Device Lead during an MRI to Reduce Lead Heating and the Risks of MRI-Induced Stimulation; U.S. patent application Ser. No. 12/257,245, filed Oct. 23, 2008, of Min, entitled “Systems and Methods for Disconnecting Electrodes of Leads of Implantable Medical Devices during an MRI to Reduce Lead Heating while also providing RF Shielding”; and U.S. patent application Ser. No. 12/270,768, of Min et al., filed Nov. 13, 2008, entitled “Systems And Methods For Reducing RF Power or Adjusting Flip Angles During an MRI For Patients with Implantable Medical Devices.”

At least some of these techniques are directed to installing RF filters, such as inductive (L) filters or inductive-capacitive (LC) filters, within the leads for use in filtering signals at frequencies associated with the RF fields of MRIs. It is particularly desirable to select or control of the inductance (L), parasitic capacitance (Cs) and parasitic resistance (Rs) of such devices to attain a high target impedance (e.g. at least 1000 ohms) at RF to achieve effective heat reduction. See, for example, U.S. patent application Ser. No. 11/955,268, filed Dec. 12, 2007, of Min, entitled “Systems and Methods for Determining Inductance and Capacitance Values for use with LC Filters within Implantable Medical Device Leads to Reduce Lead Heating during an MRI”; and U.S. patent application Ser. No. 12/325,945, of Min et al., filed Dec. 1, 2008, entitled “Systems and Methods for Selecting Components for Use in RF Filters within Implantable Medical Device Leads based on Inductance, Parasitic Capacitance and Parasitic Resistance.”

U.S. patent application Ser. No. ______, of Min et al., entitled “Implantable Medical Device Lead Incorporating Insulated Coils Formed as Inductive Bandstop Filters to Reduce Lead Heating During MRI” (A09P1042) describes leads wherein a portion of the tip and ring conductors of the leads are formed as insulated coils to function as inductive bandstop filters for filtering RF signals of MRIs. That is, the insulated coil portions of the conductors are configured to provide high impedance at RF.

Although these techniques are helpful in reducing lead heating due to MRI fields, there is room for further improvement. In particular, it has been found that any coiling of excess lead length by the clinician during device implant can affect the amount of heat reduction achieved using RF filtering elements. In this regard, following implant of the distal ends of leads into heart chambers, and prior to connection of the proximal ends of the leads into the pacemaker or ICD being implanted, there may be some excess lead length. Clinicians often wrap or coil the excess lead length around or under the pacemaker or ICD prior to connecting the leads to the device. It has been found that this can negate the efficacy of heat reduction features in leads, in some cases resulting in an increase of over 30 degrees Celsius (C) as compared to leads not coiled around or under the device. Herein, the interference in heat reduction caused by wrapping the lead around or under the device is referred to as the “coiling effect.”

It is believed that the increase in heat may be due to a shunt capacitance between the proximal portions of the lead that are wrapped around or under the device and the housing of the device itself (particularly when proximal portions of the conductors within the leads are configured as insulated coils to operate as RF bandstop filters.) As noted, a high target impedance at RF is desired to reduce heating due to the RF fields of the MRI. Insofar as leads with insulated tip or ring conductors are concerned (i.e. insulated co-radial or co-axial leads), the actual impedance achieved depends, in part, on the inductance (L) and the parasitic capacitance and resistance (Cs, Rs) of the insulated conductors. Coiling a lead around or under a device appears to add a shunt capacitance to the coiled portion of the lead due to proximity with the metallic case of the device, which adversely affects the resulting L, Cs and Rs values and reduces the impedance and hence allows for greater unwanted heating during MRIs.

Accordingly, it would be desirable to provide improved lead designs that achieve greater heat reduction during MRIs, at least in part by reducing or counteracting the “coiling effect.” Various aspects of the invention are directed to this end.

### SUMMARY OF THE INVENTION

In accordance with various exemplary embodiments of the invention, a lead is provided for use with an implantable medical device for implant within a patient wherein the lead includes: an electrode for placement adjacent patient tissues; a conductor operative to route signals along the lead between the electrode and the implantable medical device, with a portion of the conductor formed as an insulated coil and configured to function as an inductive bandstop filtering element for bandstop filtering of RF fields; and a conducting sheath surrounding at least a portion of the conductor.

The conducting sheath may be, for example, a metal mesh or a conducting polymer tube including non-ferrous metal powders. The sheath may be formed along at least a proximal portion of the lead or, preferably, along substantially its entire length. The insulated coil portion of the conductor may be formed, for example, along the entire length of the lead (continuously or in segments), or at its distal end, or at both the distal end and the proximal end of the lead. These are just some examples. By providing a conducting sheath along all or at least part of the lead, shielding of (and/or suppression of) induced currents is achieved so as to reduce electromagnetic coupling into enclosed conductors. In some examples, the conductive sheath is sub-divided into ¼ wavelength segments distributed along the entire length of the lead. By providing a conductive sheath with segments of about ¼ wavelength along the entire lead, it is believed that the induced currents from RF fields are greatly suppressed so that relatively little current can flow along the conductors enclosed by the sheath, therefore reducing heating at tip and ring electrodes. Moreover, by providing a conducting sheath around at least the proximal portion of the lead, shielding is provided to help reduce or counteract the aforementioned “coiling effect.” In particular, the conductive sheath may help to reduce or eliminate shunt capacitance between the insulated coil portion of the conductor and any external conducting structures, such as the housing of the implantable device. It is believed that reducing or eliminating the shunt capacitance has the effect, depending upon the relative proximity of the external conducting structures, of reducing heating within the lead due to strong RF fields, at least as compared to unshielded leads.

In an illustrative embodiment, wherein the lead is for use with a pacemaker or ICD, the lead is a co-axial bipolar lead having an inner conductor leading to a tip electrode at a distal end of the lead and also having an outer ring conductor leading to a ring electrode at the distal end of the lead. Both the inner and outer conductors are formed as insulated coils to function as inductive bandstop filters at RF signal frequencies. The conducting sheath generally extends along the entire lead length but is formed of several sections or segments. Each section or segment is preferred to be about ¼ wavelength (based on the wavelengths of current flowing in lead conductors in the presence of MRI RF fields or other strong magnetic fields.) Alternatively, the conducting sheath extends along just the proximal end of the lead, particularly along those portions of the lead that might be wrapped around or under the pacemaker or ICD. As such, the conducting sheath helps prevent shunt capacitance between the proximal end of the outer (i.e. ring) insulated conducting coil of the lead and the housing of the pacemaker or ICD.

In the illustrative embodiment, the L, Cs and Rs values of the inductive bandstop filter portions of the inner and outer conductors of the lead are selected or controlled to achieve a high target impedance at RF. Preferably, the filter portions in combination with the conductive sheath allow the lead to achieve an impedance of 1000 ohms or more in the presence of RF fields generated by an MRI, particularly RF fields operating at about 64 MHz or 128 MHz.

The conductive sheath is well-suited for use with coaxial, co-radial or cable bipolar/unipolar cardiac pacing/sensing leads for use with pacemakers and ICDs but also be employed in connection with other cardiac pacing/sensing leads, or other combined structure leads for use with other implantable medical devices.

### BRIEF DESCRIPTION OF THE DRAWINGS

The above and further features, advantages and benefits of the invention will be apparent upon consideration of the descriptions herein taken in conjunction with the accompanying drawings, in which:

FIG. 1 is a stylized representation of an MRI system along with a patient with a pacer/ICD implanted therein with RV and LV leads employing conductive sheathes at their proximal ends and also illustrating the coiling of the proximal ends of the leads around the pacer/ICD;

FIG. 2 is a block diagram, partly in schematic form, illustrating a co-radial bipolar lead for use with the pacer/ICD of FIG. 1 wherein a conducting sheath is provided along the entire length of the lead (and particularly around insulated coil bandstop filters formed at the proximal end of the lead) to counteract the “coiling effect” so as to reduce heating of the lead during an MRI, and also illustrating a pacer/ICD connected to the lead;

FIG. 3 is a block diagram, partly in schematic form, illustrating another embodiment of the co-radial bipolar lead for use with the pacer/ICD of FIG. 1 wherein the conducting sheath incorporates ¼ wavelength segments formed along substantially the entire length of the lead;

FIG. 4 is a side cross-sectional view of a portion of an alternative coaxial implementation of the lead of FIG. 3 wherein the conducting sheath is a layer of a conducting polymer tube impregnated with non-ferrous metal powders;

FIG. 5 is an alternative implementation of the lead of FIG. 4 wherein the conducting sheath is a metal mesh formed of metal braiding;

FIG. 6 is a simplified, partly cutaway view, illustrating the pacer/ICD of FIG. 1 along with a more complete set of leads implanted in the heart of the patient; and

FIG. 7 is a functional block diagram of the pacer/ICD of FIG. 6, illustrating basic circuit elements that provide cardioversion, defibrillation and/or pacing stimulation in four chambers of the heart.

### DETAILED DESCRIPTION OF THE PREFERRED EMBODIMENTS

The following description includes the best mode presently contemplated for practicing the invention. The description is not to be taken in a limiting sense but is made merely to describe general principles of the invention. The scope of the invention should be ascertained with reference to the issued claims. In the description of the invention that follows, like numerals or reference designators will be used to refer to like parts or elements throughout.

### Overview of MRI System

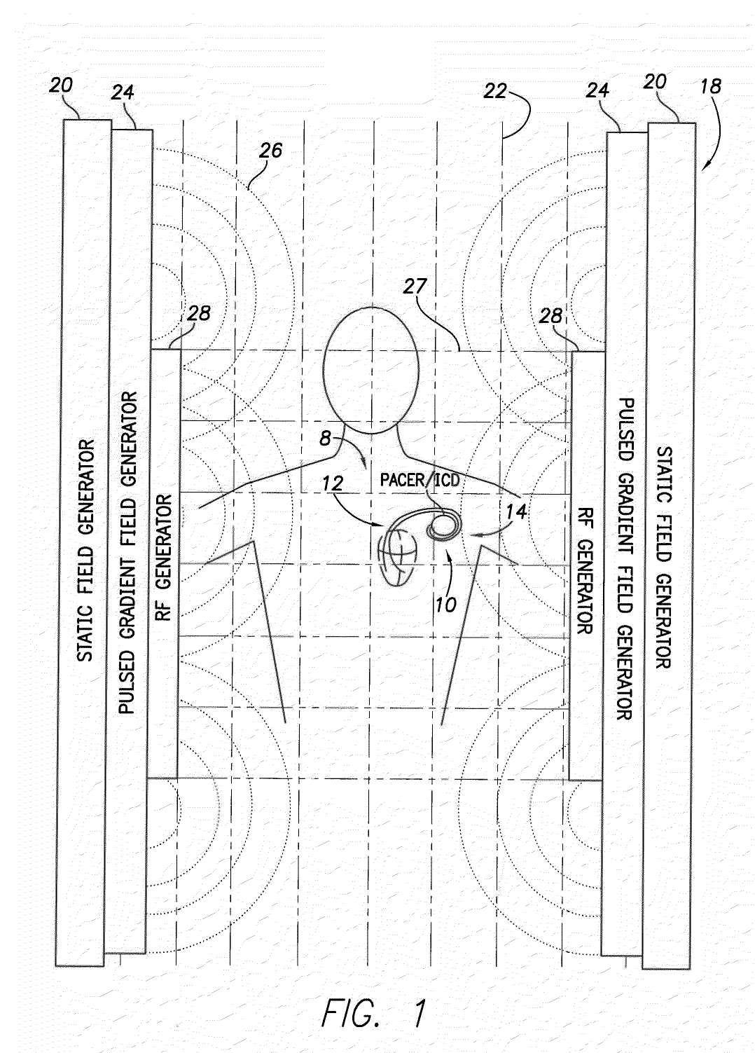

FIG. 1 illustrates an implantable medical system 8 having a pacer/ICD 10 for use with a set of bipolar pacing/sensing leads 12. In the example, proximal portions 14 of the leads have been wrapped around the pacer/ICD, as can occur if the clinician chooses to wrap excess portions of the lead around or under the device during device implant. As explained, the coiling of the lead around or under the pacer/ICD by the clinician can adversely affect heat reduction achieved by insulated coil bandstop filters within the lead (none of which are shown in FIG. 1.) As such, greater heating can occur within the lead and surrounding tissues due to the fields generated by an MRI system 18, than if the lead were not wrapped around the pacer/ICD. This is the coiling effect mentioned above.

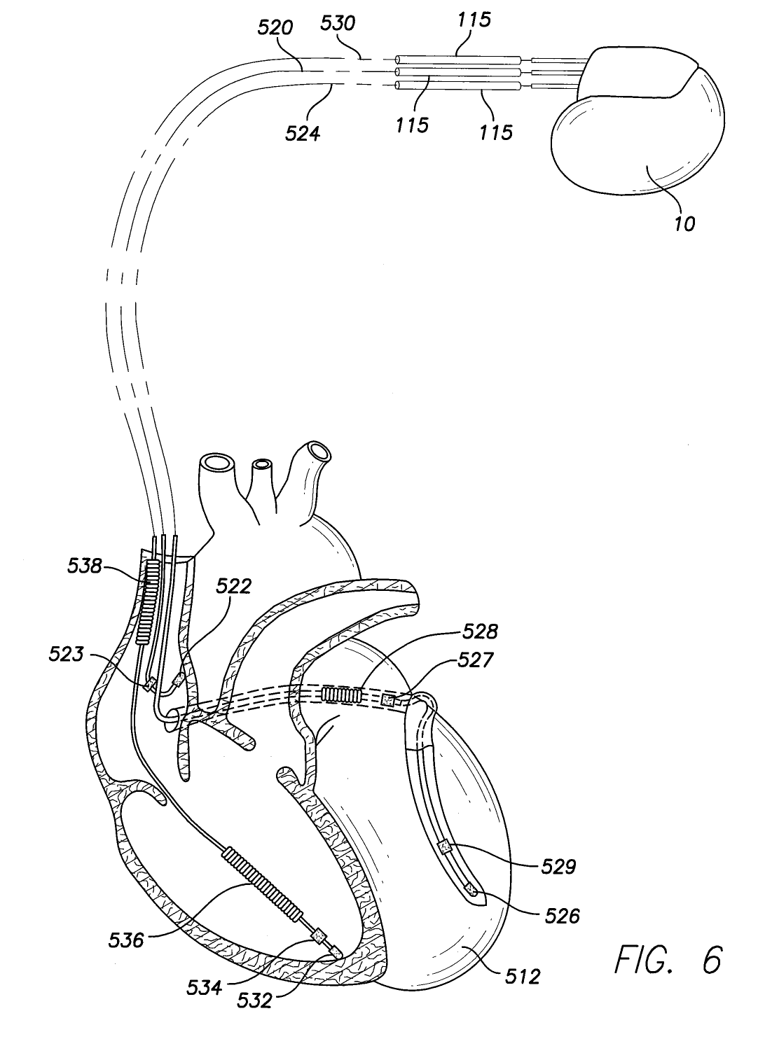

As will be explained in more detail below, leads 12 each include conducting sheaths extending along most of the lead to help suppress induced currents from RF fields along the enclosed conductors or at least along proximal portions of the lead to help reduce or eliminate shunt capacitance (or other electrical effects) between the insulated coil bandstop filters (within proximal portions of the leads) and the conducting housing of pacer/ICD. In FIG. 1, these conducting sheaths are not separately shown, as the sheaths are integral with the lead. Note also that in FIG. 1 only two leads are shown, a right ventricular (RV) lead and a left ventricular (LV) lead. A more complete lead system is illustrated in FIG. 6, described below.

As to MRI system 18, the system includes a static field generator 20 for generating a static magnetic field 22 and a pulsed gradient field generator 24 for selectively generating pulsed gradient magnetic fields 26. The MRI system also includes an RF generator 28 for generating RF fields 27. Other components of the MRI, such as its sensing and imaging components are not shown. MRI systems and imaging techniques are well known and will not be described in detail herein. For exemplary MRI systems see, for example, U.S. Pat. No. 5,063,348 to Kuhara, et al., entitled “Magnetic Resonance Imaging System” and U.S. Pat. No. 4,746,864 to Satoh, entitled “Magnetic Resonance Imaging System.” Note that the fields shown in FIG. 1 are stylized representations of MRI fields intended merely to illustrate the presence of the fields. Actual MRI fields generally have far more complex patterns.

Hence, the leads of pacer/ICD 10 include conducting sheaths installed therein for use in reducing lead heating during MRI procedures such as heating that may be due, at least in part, due to the coiling effect.

### Shielded Lead Examples

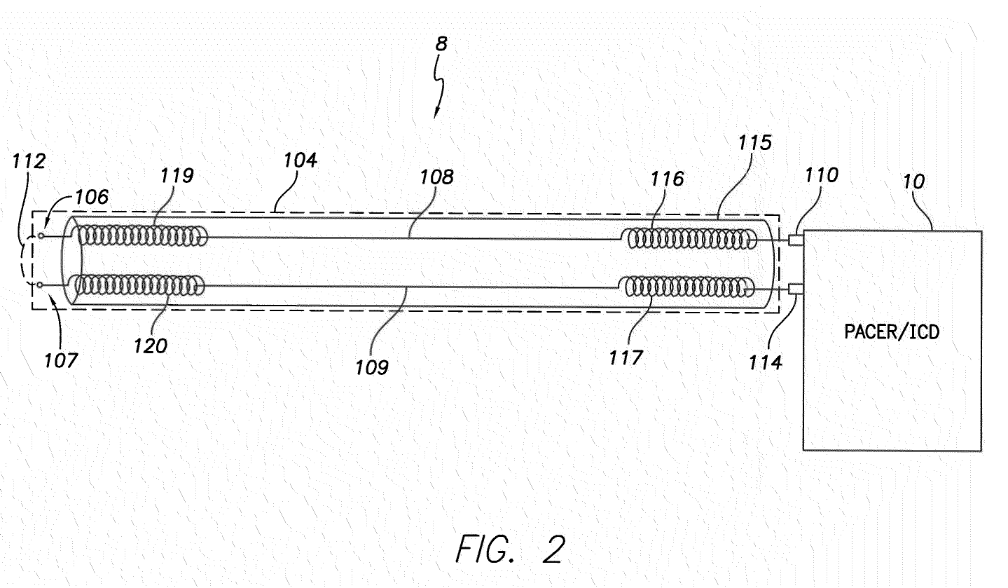

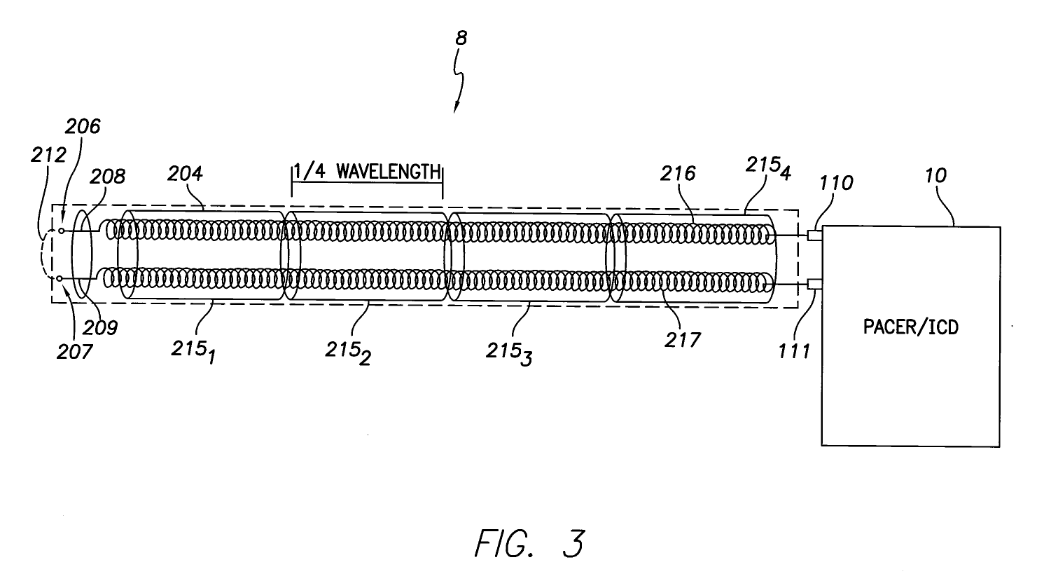

FIG. 2 illustrates implantable system 8 having a pacer/ICD or other implantable medical device 10 with a bipolar co-radial lead 104. The bipolar lead includes a tip electrode 106 electrically connected to the pacer/ICD via a tip conductor 108 coupled to a tip connector or terminal 110 of the pacer/ICD. (The tip electrode may be, for example, a pacing/sensing electrode or a high-voltage defibrillation electrode.) Conductor 108 includes, in this particular example, a first insulated coil portion 116 at the proximal of the lead, formed as an inductive bandstop filter for filtering RF signals associated with MRIs. Conductor 108 also includes, in this example, a second insulated coil portion 119 at the distal end of the lead, also formed as an inductive bandstop filter. The bipolar lead also includes a ring electrode 107 electrically connected to the pacer/ICD via a ring conductor 109 coupled to a ring connector or terminal 111 of the pacer/ICD. The ring conductor includes a proximal insulated coil portion 117 and a distal insulated coil portion 120. As with the coiled portions of the tip conductor, the insulated coils of the ring conductor are provided to function as inductive bandstop filters for filtering RF signals associated with MRIs. The configuration and electrical parameters of the coiled portions of the lead conductors can be set so as to impede the conduction of signals at selected RF frequencies, such as 64 MHz or at 128 MHz. In other examples, the coiled portion of the tip and ring conductors can be positioned elsewhere along the length of the lead (or along substantially the entire length of the lead as shown in FIG. 3 described below.)

Depending upon the particular implementation, during pacing/sensing, the tip electrode may be more negative than the ring, or vice versa. A conducting path 112 between tip electrode 106 and ring electrode 107 is provided through patient tissue (typically cardiac tissue.)

A conducting sheath 115 is provided, in this example, along substantially most of lead 104 surrounding both the proximal and distal insulated coils. The conducting sheath can be, for example, formed of a metal mesh or a layer of a conducting polymer tube incorporating non-ferrous metal powders or other suitable material. The conducting sheath can be embedded inside insulation tubing of the lead or on the interface of lead exposed to patient tissues/fluids. An alternative embodiment where the sheath is formed of ¼ wavelength segments is discussed below.

In an embodiment where a conducting sheath is provided around at least the proximal ends of the tip and ring conductors, particularly around the proximal coils, electromagnetic shielding is thereby provided to help reduce heat during MRIs. As explained, the conductive sheath appears to reduce or eliminate further coupling or shunt capacitance between the insulated coil portions of tip and ring conductors and the housing of the implantable device, especially if the proximal end of the lead is wrapped around or under the device during implant by the clinician (as in FIG. 1.) It is believed that shielding has the effect of suppressing induced currents on the enclosed conductors (at least when the sheath is formed along most of lead). It is also believed that shielding has the effect of reducing or eliminating shunt capacitance, at least when the sheath if formed at the proximal end of the lead and further has the effect, depending upon the relative proximity of the external conducting structures, of reducing heating within the lead due to strong RF fields, such as those used during MRI procedures. As explained above, such heating can damage patient tissue and interfere with pacing and sensing. Nevertheless, regardless of the precise reason for its efficacy, the conducting sheath has a beneficial effect during MRIs, at least as compared to similarly configured leads without such shielding when such leads are coiled around or under a pacer/ICD.

Additionally, although not specifically shown, the lead may include one or more switches or additional RF filters mounted elsewhere along the lead to further block or filter RF signals during MRIs to further reduce lead heating during MRIs or in the presence of other strong RF fields. Also, note that conductive sheath 115 can be electrically connected to the ring terminal electrode (or other can electrode terminals of the device housing.) In other implementations, the sheath is not electrically connected to the device housing.

FIG. 3 illustrates an alternative implementation wherein a co-radial lead 204 is provided with a sheath 215 that extends substantially along the entire length of the lead and is sub-divided into ¼ wavelength segments. As with lead 104 of FIG. 2, the lead of FIG. 3 includes a tip electrode 206 connected to the pacer/ICD via a tip conductor 208 coupled to tip terminal 210, wherein the tip conductor includes an insulated coiled portion 216 provided to function as an inductive bandstop filter for filtering RF signals associated with MRIs. In this example, insulated coil 216 extends along substantially the entire length of the lead including the proximal end of the lead. The lead also includes a ring electrode 207 electrically connected to the pacer/ICD via a ring conductor 209 coupled to ring terminal 111, wherein the ring conductor includes an insulated coil portion 217 extending along the length of the lead including the proximal end of the lead. A conducting path 212 is provided between tip electrode 206 and ring electrode 207 through patient tissue.

Insofar as the sheath of FIG. 3 is concerned, sheath 215 is composed of ¼ wavelength segments based on the wavelength of current flowing along the conductors within the leads in MRI RF fields or other strong magnetic fields. That is, the length of each segment of the sheath is preferably set to about one quarter of a wavelength of the expected current. For an MRI, the wavelengths of RF current induced in the leads varies typically about the length of the lead or are integer multiples or fractions thereof, which depends on, e.g., lead structure, lead length and MRI RF frequencies. If the wavelength of the induced currents is expected to be about equal to the length of the lead, then four sheath segments 215₁-215₄ may be provided (as shown) with: a first segment 215₁ extending from the proximal end of the lead to about a quarter point of the lead; a second segment 215₂ extending from about the quarter point of the lead to the half point of the lead; a third segment 215₃ extending from about the half-point of the lead to the three-quarter point of the lead; and a fourth segment 215₄ extending from about the three-quarter point of the lead to the near the distal end of the lead; (i.e. at or near the location of the ring electrode.) The segments need not be exactly quarter wavelengths.

As with the conducting sheath of FIG. 2, the segments of the sheath of FIG. 3 can be formed of a metal mesh or a conducting polymer tube incorporating non-ferrous metal powders or other suitable material sufficient to reduce heat during MRIs by blocking shunt capacitances or for other reasons. The sheath segments are embedded in insulation tubing and are separated by insulation material. In one example, the separation or spacing between each pair of adjacent segments is about 2-6 millimeters (mm.)

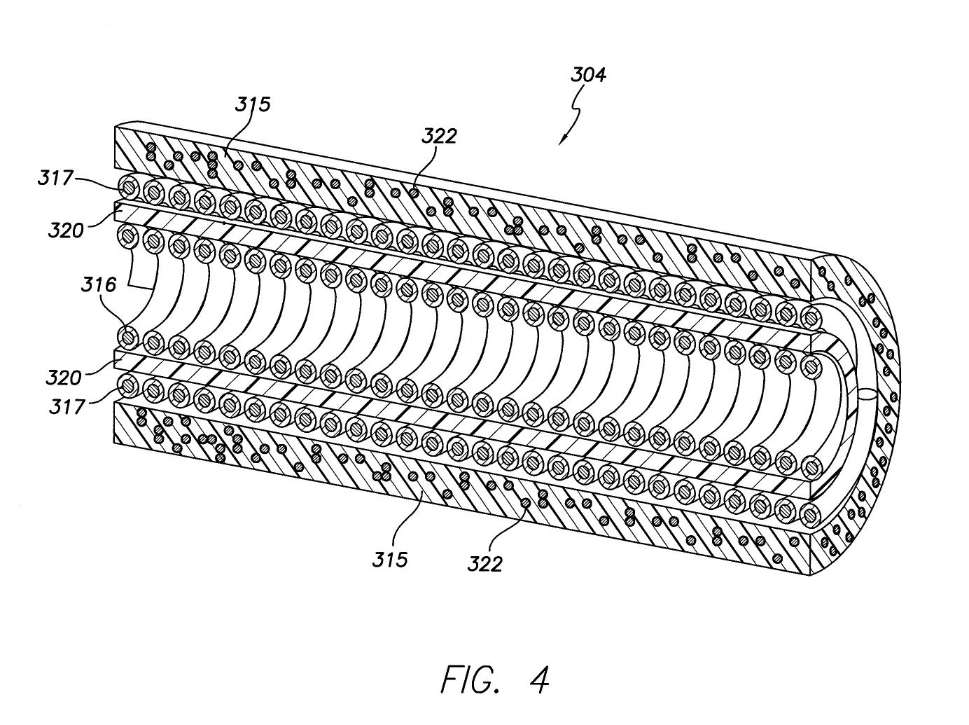

FIG. 4 illustrates a coaxial implementation of the shielded lead wherein the conductive sheath is a layer of a conducting polymer tube impregnated with non-ferrous metal powders. The conducting polymer tube can be either embedded inside insulation layers (silicone rubber, Optim, silicone rubber polyurethane copolymer (“SPC”), or polyurethane on the outer layer exposed to patient tissues/fluids. (Optim is a registered trademark of Pacesetter, Inc. DBA St. Jude Medical Cardiac Rhythm Management Division. Optim refers to a silicone-polyurethane co-polymer insulation created specifically for cardiac leads. The new material blends the biostability and flexibility of silicone with the durability, lubricity and abrasion-resistance of polyurethane.) Alternatively, materials such as tetrafluoroethylene (“ETFE”), polytetrafluoroethylene (“PTFE”), silicone rubber, silicone rubber polyurethane copolymer (“SPC”) can be used. In particular, ETFE or PTFE can be used for wire or coil coatings (such as around the conducting coils). Silicone rubber or SPC can be used as part of the conducting sheath tubing (impregnated, e.g., with non-ferrous metal powders.)

Coaxial lead 304 includes an insulated coiled tip conductor 316 surrounded by an insulated coiled ring conductor 317 (wherein the insulated coils are configured to function as inductive bandstop filters for filtering RF signals.) An intermediate insulator 320 is positioned between the tip and ring coils. The intermediate insulator may be, for example, formed of ETFE, PTFE or silicone or other suitable materials (as mentioned above.) Carbon composite can be added to the polymer insulator. The conducting sheath 315 encloses both the tip and ring insulated coils, as shown. In this example, the sheath is a conducting polymer tube incorporating non-ferrous metal powders 322. Exemplary non-ferrous metal powders that may be used include gold, platinum, iridium, and a nonmagnetic, nickel-cobalt-chromium-molybdenum alloy commonly referred to using the tradename MP35N.

Otherwise routine testing and experimentation may be performed to determine preferred parameters for configuring the sheath—such as its inner and outer diameters and the size and density of the non-ferrous metal powders employed therein—for use in a particular lead so as to achieve adequate reduction in lead temperatures during an MRI or in the presence of other sources of strong RF fields. The density of the powder can be 90% or higher.

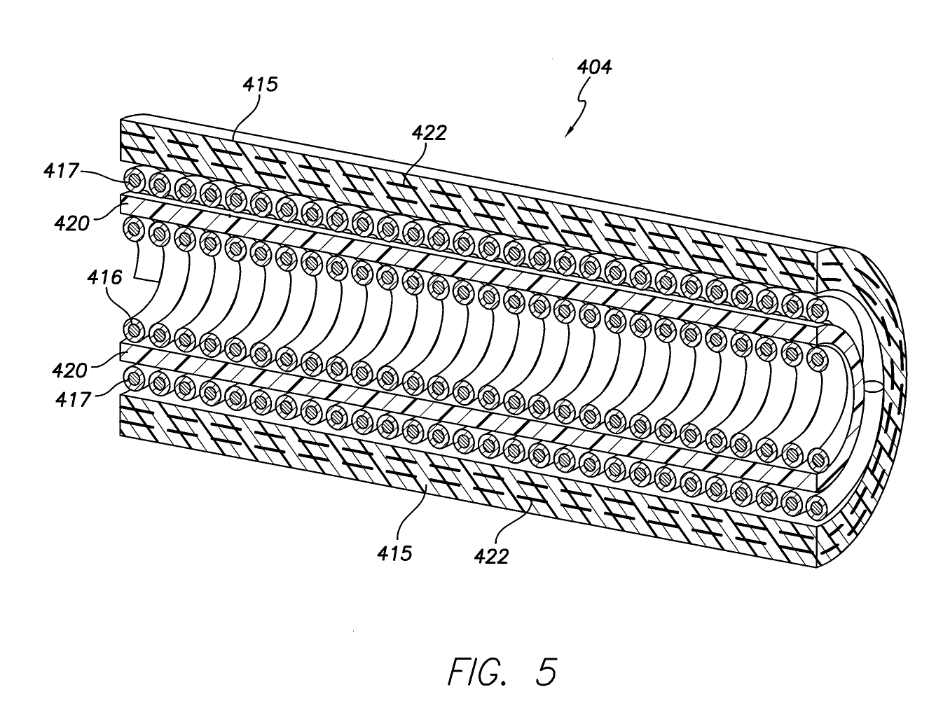

FIG. 5 illustrates another coaxial implementation of the shielded lead, wherein the conductive sheath includes a metal mesh or metal braid. Coaxial lead 404 includes an insulated coil tip conductor 416 surrounded by an insulated coil ring conductor 417 (wherein the insulated coils are again configured to function as inductive bandstop filters for filtering RF signals.) An intermediate insulator 420 is positioned between the tip and ring coils. The conducting sheath 415 again encloses both the tip and ring coils. In this example, the sheath includes a metal mesh or braiding 422. In-vitro tests have demonstrated that a mesh lead using braiding wires reduced RF heating about 50% over the control leads (without a mesh shielding) within a gel phantom. Modeling results showed more heating reduction can be achieved with the conducting sheath sections formed of about ¼ wavelength segments.

The various configurations described above can be exploited for use with a wide variety of implantable medical systems. For the sake of completeness, a detailed description of an exemplary pacer/ICD and lead system will now be provided.

### Exemplary Pacer/ICD/Lead System

FIG. 6 provides a simplified diagram of the pacer/ICD of FIG. 1, which is a dual-chamber stimulation device capable of treating both fast and slow arrhythmias with stimulation therapy, including cardioversion, defibrillation, and pacing stimulation. To provide atrial chamber pacing stimulation and sensing, pacer/ICD 10 is shown in electrical communication with a heart 512 by way of a left atrial lead 520 having an atrial tip electrode 522 and an atrial ring electrode 523 implanted in the atrial appendage. Pacer/ICD 10 is also in electrical communication with the heart by way of a right ventricular lead 530 having, in this embodiment, a ventricular tip electrode 532, a right ventricular ring electrode 534, a right ventricular (RV) coil electrode 536. Typically, the right ventricular lead 530 is transvenously inserted into the heart so as to place the RV coil electrode 536 in the right ventricular apex. Accordingly, the right ventricular lead is capable of receiving cardiac signals, and delivering stimulation in the form of pacing and shock therapy to the right ventricle. Conducting sheaths 115, configured as described above, are positioned along the lead including (as shown in this particular example) proximal portions of leads 520 and 530 so as to reduce lead heating. Note that, in the figure, portions of the leads between the pacer/ICD and the heart are shown in phantom lines so as to more clearly illustrate the sheaths. [Portions of the leads positioned internal the heart are shown without phantom lines so as to more clearly illustrate the various pacing/shocking electrodes.]

To sense left atrial and ventricular cardiac signals and to provide left chamber pacing therapy, pacer/ICD 10 is coupled to a “coronary sinus” lead 524 designed for placement in the “coronary sinus region” via the coronary sinus os for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus. Accordingly, an exemplary coronary sinus lead 524 is designed to receive atrial and ventricular cardiac signals and to deliver left ventricular pacing therapy using at least a left ventricular tip electrode 526 and a left ventricular ring electrode 529 and to deliver left atrial pacing therapy using at least a left atrial ring electrode 527, and shocking therapy using at least an SVC coil electrode 528. As with leads 520 and 530, a conducting sheath, configured as described above, is also positioned along a proximal portion of lead 524.

With this lead configuration, biventricular pacing can be performed. Although only three leads are shown in FIG. 6, it should also be understood that additional stimulation leads (with one or more pacing, sensing and/or shocking electrodes) may be used in order to efficiently and effectively provide pacing stimulation to the left side of the heart or atrial cardioversion and/or defibrillation.

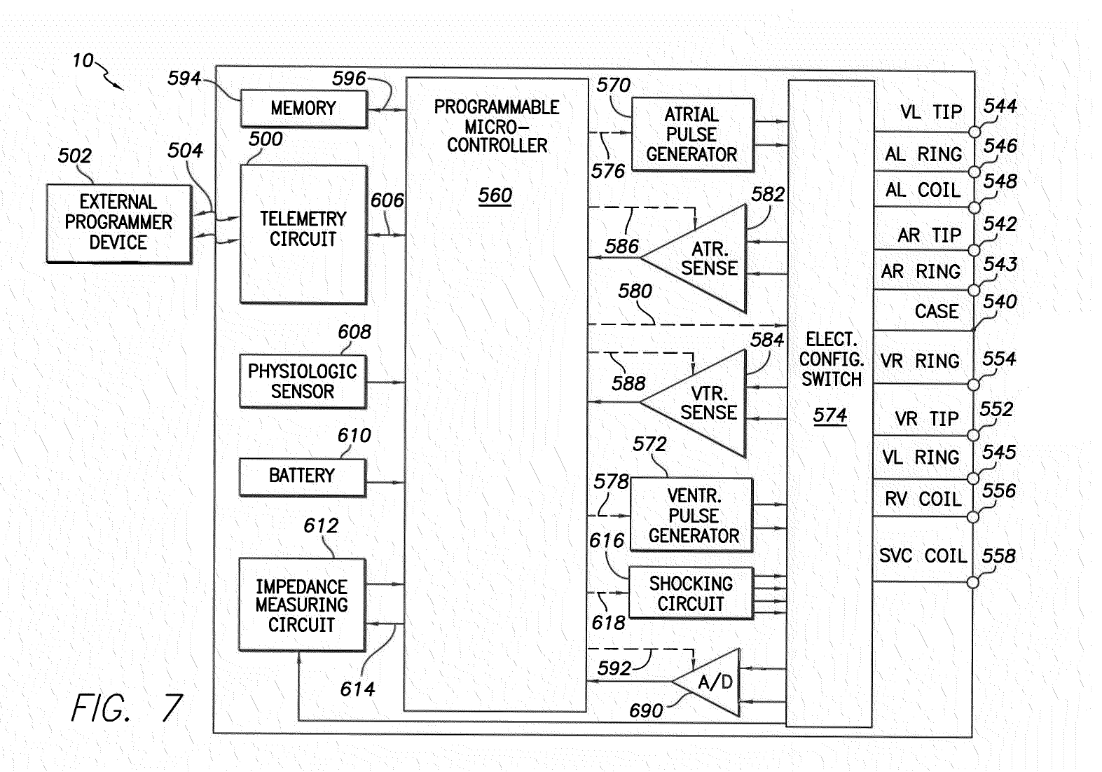

A simplified block diagram of internal components of pacer/ICD 10 is shown in FIG. 7. While a particular pacer/ICD is shown, this is for illustration purposes only, and one of skill in the art could readily duplicate, eliminate or disable the appropriate circuitry in any desired combination to provide a device capable of treating the appropriate chamber(s) with cardioversion, defibrillation and pacing stimulation as well as providing for the aforementioned apnea detection and therapy.

The housing 540 for pacer/ICD 10, shown schematically in FIG. 7, is often referred to as the “can”, “case” or “case electrode” and may be programmably selected to act as the return electrode for all “unipolar” modes. The housing 540 may further be used as a return electrode alone or in combination with one or more of the coil electrodes, 528, 536 and 538, for shocking purposes. The housing 540 further includes a connector (not shown) having a plurality of terminals, 542, 543, 544, 545, 546, 548, 552, 554, 556 and 558 (shown schematically and, for convenience, the names of the electrodes to which they are connected are shown next to the terminals). As such, to achieve right atrial sensing and pacing, the connector includes at least a right atrial tip terminal (A_{R} TIP) 542 adapted for connection to the atrial tip electrode 522 and a right atrial ring (A_{R} RING) electrode 543 adapted for connection to right atrial ring electrode 523. To achieve left chamber sensing, pacing and shocking, the connector includes at least a left ventricular tip terminal (V_{L} TIP) 544, a left ventricular ring terminal (V_{L} RING) 545, a left atrial ring terminal (A_{L} RING) 546, and a left atrial shocking terminal (A_{L} COIL) 548, which are adapted for connection to the left ventricular ring electrode 526, the left atrial tip electrode 527, and the left atrial coil electrode 528, respectively. To support right chamber sensing, pacing and shocking, the connector further includes a right ventricular tip terminal (V_{R} TIP) 552, a right ventricular ring terminal (V_{R} RING) 554, a right ventricular shocking terminal (R_{V} COIL) 556, and an SVC shocking terminal (SVC COIL) 558, which are adapted for connection to the right ventricular tip electrode 532, right ventricular ring electrode 534, the RV coil electrode 536, and the SVC coil electrode 538, respectively.

At the core of pacer/ICD 10 is a programmable microcontroller 560, which controls the various modes of stimulation therapy. As is well known in the art, the microcontroller 560 (also referred to herein as a control unit) typically includes a microprocessor, or equivalent control circuitry, designed specifically for controlling the delivery of stimulation therapy and may further include RAM or ROM memory, logic and timing circuitry, state machine circuitry, and I/O circuitry. Typically, the microcontroller 560 includes the ability to process or monitor input signals (data) as controlled by a program code stored in a designated block of memory. The details of the design and operation of the microcontroller 560 are not critical to the invention. Rather, any suitable microcontroller 560 may be used that carries out the functions described herein. The use of microprocessor-based control circuits for performing timing and data analysis functions are well known in the art.

As shown in FIG. 7, an atrial pulse generator 570 and a ventricular pulse generator 572 generate pacing stimulation pulses for delivery by the right atrial lead 520, the right ventricular lead 530, and/or the coronary sinus lead 524 via an electrode configuration switch 574. It is understood that in order to provide stimulation therapy in each of the four chambers of the heart, the atrial and ventricular pulse generators, 570 and 572, may include dedicated, independent pulse generators, multiplexed pulse generators or shared pulse generators. The pulse generators, 570 and 572, are controlled by the microcontroller 560 via appropriate control signals, 576 and 578, respectively, to trigger or inhibit the stimulation pulses.

The microcontroller 560 further includes timing control circuitry (not separately shown) used to control the timing of such stimulation pulses (e.g., pacing rate, atrio-ventricular (AV) delay, atrial interconduction (A-A) delay, or ventricular interconduction (V-V) delay, etc.) as well as to keep track of the timing of refractory periods, blanking intervals, noise detection windows, evoked response windows, alert intervals, marker channel timing, etc., which is well known in the art. Switch 574 includes a plurality of switches for connecting the desired electrodes to the appropriate I/O circuits, thereby providing complete electrode programmability. Accordingly, the switch 574, in response to a control signal 580 from the microcontroller 560, determines the polarity of the stimulation pulses (e.g., unipolar, bipolar, combipolar, etc.) by selectively closing the appropriate combination of switches (not shown) as is known in the art.

Atrial sensing circuits 582 and ventricular sensing circuits 584 may also be selectively coupled to the right atrial lead 520, coronary sinus lead 524, and the right ventricular lead 530, through the switch 574 for detecting the presence of cardiac activity in each of the four chambers of the heart. Accordingly, the atrial (ATR. SENSE) and ventricular (VTR. SENSE) sensing circuits, 582 and 584, may include dedicated sense amplifiers, multiplexed amplifiers or shared amplifiers. The switch 574 determines the “sensing polarity” of the cardiac signal by selectively closing the appropriate switches, as is also known in the art. In this way, the clinician may program the sensing polarity independent of the stimulation polarity. Each sensing circuit, 582 and 584, preferably employs one or more low power, precision amplifiers with programmable gain and/or automatic gain control and/or automatic sensitivity control, bandpass filtering, and a threshold detection circuit, as known in the art, to selectively sense the cardiac signal of interest. The automatic gain and/or sensitivity control enables pacer/ICD 10 to deal effectively with the difficult problem of sensing the low amplitude signal characteristics of atrial or ventricular fibrillation. The outputs of the atrial and ventricular sensing circuits, 582 and 584, are connected to the microcontroller 560 which, in turn, are able to trigger or inhibit the atrial and ventricular pulse generators, 570 and 572, respectively, in a demand fashion in response to the absence or presence of cardiac activity in the appropriate chambers of the heart.

For arrhythmia detection, pacer/ICD 10 utilizes the atrial and ventricular sensing circuits, 582 and 584, to sense cardiac signals to determine whether a rhythm is physiologic or pathologic. As used herein “sensing” is reserved for the noting of an electrical signal, and “detection” is the processing of these sensed signals and noting the presence of an arrhythmia. The timing intervals between sensed events (e.g., P-waves, R-waves, and depolarization signals associated with fibrillation which are sometimes referred to as “Fib-waves”) are then classified by the microcontroller 560 by comparing them to a predefined rate zone limit (i.e., bradycardia, normal, atrial tachycardia, atrial fibrillation, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, antitachycardia pacing, cardioversion shocks or defibrillation shocks).

Cardiac signals are also applied to the inputs of an analog-to-digital (A/D) data acquisition system 590. The data acquisition system 590 is configured to acquire intracardiac electrogram signals, convert the raw analog data into a digital signal, and store the digital signals for later processing and/or telemetric transmission to an external device 602. The data acquisition system 590 is coupled to the right atrial lead 520, the coronary sinus lead 524, and the right ventricular lead 530 through the switch 574 to sample cardiac signals across any pair of desired electrodes. The microcontroller 560 is further coupled to a memory 594 by a suitable data/address bus 596, wherein the programmable operating parameters used by the microcontroller 560 are stored and modified, as required, in order to customize the operation of pacer/ICD 10 to suit the needs of a particular patient. Such operating parameters define, for example, pacing pulse amplitude or magnitude, pulse duration, electrode polarity, rate, sensitivity, automatic features, arrhythmia detection criteria, and the amplitude, waveshape and vector of each shocking pulse to be delivered to the patient's heart within each respective tier of therapy. Other pacing parameters include base rate, rest rate and circadian base rate.

Advantageously, the operating parameters of the implantable pacer/ICD 10 may be non-invasively programmed into the memory 594 through a telemetry circuit 600 in telemetric communication with an external device 602, such as a programmer, transtelephonic transceiver or a diagnostic system analyzer, or a bedside monitoring system. The telemetry circuit 600 is activated by the microcontroller by a control signal 606. The telemetry circuit 600 advantageously allows IEGMs and other electrophysiological signals and/or hemodynamic signals and status information relating to the operation of pacer/ICD 10 (as stored in the microcontroller 560 or memory 594) to be sent to the external programmer device 602 through an established communication link 604.

Pacer/ICD 10 further includes an accelerometer or other physiologic sensor 608, commonly referred to as a “rate-responsive” sensor because it is typically used to adjust pacing stimulation rate according to the exercise state of the patient. However, the physiological sensor 608 may further be used to detect changes in cardiac output, changes in the physiological condition of the heart, or diurnal changes in activity (e.g., detecting sleep and wake states) and to detect arousal from sleep. Accordingly, the microcontroller 560 responds by adjusting the various pacing parameters (such as rate, AV Delay, V-V Delay, etc.) at which the atrial and ventricular pulse generators, 570 and 572, generate stimulation pulses. While shown as being included within pacer/ICD 10, it is to be understood that the physiologic sensor 608 may also be external to pacer/ICD 10, yet still be implanted within or carried by the patient. A common type of rate responsive sensor is an activity sensor incorporating an accelerometer or a piezoelectric crystal, which is mounted within the housing 540 of pacer/ICD 10. Other types of physiologic sensors are also known, for example, sensors that sense the oxygen content of blood, respiration rate and/or minute ventilation, pH of blood, ventricular gradient, etc.

The pacer/ICD additionally includes a battery 610, which provides operating power to all of the circuits shown in FIG. 7. The battery 610 may vary depending on the capabilities of pacer/ICD 10. If the system only provides low voltage therapy, a lithium iodine or lithium copper fluoride cell may be utilized. For pacer/ICD 10, which employs shocking therapy, the battery 610 must be capable of operating at low current drains for long periods, and then be capable of providing high-current pulses (for capacitor charging) when the patient requires a shock pulse. The battery 610 must also have a predictable discharge characteristic so that elective replacement time can be detected. Accordingly, pacer/ICD 10 is preferably capable of high voltage therapy and appropriate batteries.

As further shown in FIG. 7, pacer/ICD 10 is shown as having an impedance measuring circuit 612 which is enabled by the microcontroller 560 via a control signal 614. Various uses for an impedance measuring circuit include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring respiration; and detecting the opening of heart valves, measuring lead resistance, etc. The impedance measuring circuit 612 is advantageously coupled to the switch 574 so that any desired electrode may be used.

In the case where pacer/ICD 10 is intended to operate as an implantable cardioverter/defibrillator (ICD) device, it detects the occurrence of an arrhythmia, and automatically applies an appropriate electrical shock therapy to the heart aimed at terminating the detected arrhythmia. To this end, the microcontroller 560 further controls a shocking circuit 616 by way of a control signal 618. The shocking circuit 616 generates shocking pulses of low (up to 0.5 joules), moderate (0.5-11 joules) or high energy (11 to at least 40 joules), as controlled by the microcontroller 560. Such shocking pulses are applied to the heart of the patient through at least two shocking electrodes, and as shown in this embodiment, selected from the left atrial coil electrode 528, the RV coil electrode 536, and/or the SVC coil electrode 538. The housing 540 may act as an active electrode in combination with the RV electrode 536, or as part of a split electrical vector using the SVC coil electrode 538 or the left atrial coil electrode 528 (i.e., using the RV electrode as a common electrode). Cardioversion shocks are generally considered to be of low to moderate energy level (so as to minimize pain felt by the patient), and/or synchronized with an R-wave and/or pertaining to the treatment of tachycardia. Defibrillation shocks are generally of moderate to high energy level (i.e., corresponding to thresholds in the range of 11-40 joules), delivered asynchronously (since R-waves may be too disorganized), and pertaining exclusively to the treatment of fibrillation. Accordingly, the microcontroller 560 is capable of controlling the synchronous or asynchronous delivery of the shocking pulses.

What have been described are systems and methods for use with a set of pacing/sensing leads for use with a pacer/ICD. Principles of the invention may be exploiting using other implantable systems or in accordance with other techniques. Thus, while the invention has been described with reference to particular exemplary embodiments, modifications can be made thereto without departing from the scope of the invention.

What is claimed is:

1. A lead for use with an implantable medical device for implant within a patient, the lead comprising:
an electrode for placement adjacent patient tissues;
a conductor operative to route signals along the lead between the electrode and the implantable medical device, with a portion of the conductor formed as an insulated coil and configured to function as an inductive bandstop filtering element for filtering radio-frequency (RF) fields; and
a conducting sheath surrounding at least a portion of the conductor.

2. The lead of claim 1 wherein the conducting sheath comprises a metal mesh.

3. The lead of claim 2 wherein the metal mesh comprises metal braiding.

4. The lead of claim 1 wherein the conducting sheath comprises a conducting polymer sheath.

5. The lead of claim 4 wherein the conducting polymer sheath includes non-ferrous metal powders.

6. The lead of claim 5 wherein the non-ferrous metal powders include one or more of gold, platinum, iridium, and a nonmagnetic, nickel-cobalt-chromium-molybdenum alloy.

7. The lead of claim 1 wherein the conducting sheath extends along at least a proximal portion of the conductor.

8. The lead of claim 1 wherein the conducting sheath extends along substantially most of the conductor including the proximal portion of the conductor.

9. The lead of claim 1 wherein the conducting sheath extends along substantially the entire length of the conductor from near a proximal end to near a distal end.

10. The lead of claim 1 wherein the conducting sheath is formed in segments having lengths corresponding to a quarter wavelength of currents induced within the lead by magnetic resonance imaging (MRI) fields.

11. The lead of claim 1 wherein the conducting sheath is positioned to substantially counteract any increased heating of the lead during magnetic resonance imaging (MRI) due to possible coiling of the proximal portion of the lead near the implantable medical device.

12. The lead of claim 1 wherein the insulated coil portion of the conductor is configured to provide sufficient impedance at RF signal frequencies to substantially reduce heating of the lead during magnetic resonance imaging (MRI).

13. The lead of claim 12 wherein the insulated coil portion of the conductor provides at least 1000 ohms of impedance at the RF signal frequencies of MRI fields.

14. The lead of claim 1 wherein the portion of the conductor formed as an insulated coil extends along substantially the entire lead and is surrounded by the conducting sheath.

15. The lead of claim 1 wherein the portion of the conductor formed as an insulated coil extends along at least the proximal portion of the lead and is surrounded by the conducting sheath.

16. The lead of claim 1 wherein the portion of the conductor formed as an insulated coil is positioned along at least a distal portion of the lead and is surrounded by the conducting sheath.

17. The lead of claim 1 wherein the insulated coil is insulated with one or more of: polytetrafluoroethylene (PTFE), tetrafluoroethylene (ETFE), a polymer coating or a polyimide coating.

18. The lead of claim 1 wherein the implantable device is equipped to provide cardiac defibrillation and wherein the electrode of the lead is a high-voltage defibrillation electrode.

19. The lead of claim 1 wherein the implantable device is equipped to provide cardiac pacing and wherein the electrode of the lead is a pacing electrode.

20. The lead of claim 1 wherein the conductor is a tip conductor of the lead and wherein the electrode is a tip electrode.

21. The lead of claim 20 wherein the lead further includes a ring conductor coupled to a ring electrode, the conducting sheath also surrounding at least a portion of the ring conductor.

22. The lead of claim 21 wherein a portion of the ring conductor is also formed as an insulated coil and wherein at least a portion of the ring conductor is configured to function as an inductive bandstop filtering element.

23. The lead of claim 22 wherein at least a first portion of the ring conductor formed as an insulated coil extends along a distal end of the lead and wherein at least a second portion of the ring conductor formed as an insulated coil is extends along the proximal end of the lead and wherein at least the proximal end is surrounded by the conducting sheath.

24. The lead of claim 21 wherein the lead is coaxial.

25. The lead of claim 21 wherein the lead is co-radial.

26. The lead of claim 21 wherein the lead incorporates a multi-lumen cable structure.

27. A bipolar lead for use with an implantable medical device for implant within a patient, the lead comprising:
first and second electrodes for placement adjacent patient tissues;
a first conductor operative to route signals along the lead between the first electrode and the implantable medical device, with a portion of the first conductor formed as an insulated coil to provide inductive bandstop filtering of radio-frequency (RF) fields;
a second conductor operative to route signals along the lead between the second electrode and the implantable medical device, with a portion of the second conductor also formed as an insulated coil to provide inductive bandstop filtering of RF fields; and
a conducting sheath surrounding at least portions of the first and second conductors.

28. An implantable medical system for implant within a patient comprising:
an implantable cardiac rhythm management device; and
a lead for use with the implantable medical device wherein the lead includes an electrode for placement adjacent patient tissues, a conductor for routing signals along the lead between the electrode and the implantable medical device, with a portion of the conductor formed as an insulated coil and configured to function as an inductive bandstop filtering element for filtering radio-frequency (RF) fields, and a conducting sheath surrounding at least a portion of the conductor.
